(19) United States
(12) Patent Application Publication
(10) Pub. No.: US 2016/0100611 A1
(43) Pub. Date: Apr. 14, 2016

(54) DIETARY SUPPLEMENTS AND ADDITIVES FOR LIVESTOCK

(71) Applicant: Robinson Show Cattle, LLC, Cylinder, IA (US)

(72) Inventor: JOHN D. ROBINSON, Cylinder, IA (US)

(21) Appl. No.: 14/882,709

(22) Filed: Oct. 14, 2015

Related U.S. Application Data
(60) Provisional application No. 62/063,461, filed on Oct. 14, 2014.

(51) Int. Cl. A23K 1/18; A61K 33/06; A61K 33/00; A61K 33/26; A61K 33/32; A61K 33/04; A61K 33/30; A61K 31/07; A61K 31/593; A61K 31/714; A61K 31/525; A61K 31/195; A61K 31/51; A61K 31/455; A61K 31/14; A23K 1/16; A23K 1/175; A61K 33/34

(52) U.S. Cl. CPC A23K 1/1813; A61K 33/34; A61K 33/06; A61K 33/00; A61K 33/26; A61K 33/32; A61K 33/04; A61K 33/30; A61K 31/07; A61K 31/593; A61K 31/714; A61K 31/525; A61K 31/195; A61K 31/51; A61K 31/455; A61K 31/14; A23K 1/1603; A23K 1/1609; A23K 1/1612; A23K 1/1618; A23K 1/164; A23K 1/1634; A23K 1/1646; A23K 1/1751; A23K 1/1753; A23K 1/1755; A23K 1/1756; A23K 1/1758; A23K 1/175

(57) ABSTRACT

The present invention relates to improved heath and performance of livestock produced by novel compositions and methods involving formulations provided herein. The present invention may be applied to a variety of livestock, including ruminants such as cattle and sheep.

### PRIORITY STATEMENT

This application claims priority to U.S. Provisional Patent Application No. 62/063,461, filed Oct. 14, 2014, hereby incorporated by reference in its entirety.

### FIELD OF THE INVENTION

The present invention relates to methods and compositions for increasing the health and production of livestock animals. More particularly, the present invention relates to dietary supplements for livestock, and methods for making and using them.

### BACKGROUND OF THE INVENTION

The conventional dietetic approach to feedlotting of ruminants is to feed high proportions of grain (>70%) and low proportions of roughage (<20%) in the diet. The aim of this strategy is to maximize the digestibility of the grain and its constituent nutrients including starch and proteins in the rumen. However, there is considerable variation in the starch content of different grains. For example, wheat contains about 70-75% starch, corn and sorghum 68-72% starch, and oats and barley 56-60% starch. Further, there are large differences in the rumen digestibility of starch for the different grains. For example, the starch sources present in wheat and barley are highly digested in the rumen (84-90%) whereas that of sorghum is much lower (60-65%). Such variation in starch content and rumen digestibility results in major differences in feedlot performance and in turn this has significant implications for profitability.

When assessing the nutrient requirements of animals in feedlots it is also necessary to take into account their stage of physiological growth and maturity. For example, at the beginning of beef feedlotting cattle invariably undergo an adaptation phase which involves transition from free range grazing to intensive feedlotting, and commensurately, there is a major change in nutrient supply from a forage to a grain based diet. In effect, this creates an enormous metabolic challenge for the animal and invariably results in compensatory phases of growth occurring. This generally occurs in the first 40-50 days of feedlotting and during this period there is an additional demand for essential amino acids, essential fatty acids and glucose to satisfy intestinal growth and development, which increases during this compensatory/adaptation phase. A further example where different supplies of nutrient are required is in fat deposition and in particular the accumulation of intramuscular fat—a process commonly referred to as marbling.

Efficient rumen fermentation in ruminants is important to maximizing animal performance, including feed intake, feed efficiency, production of milk, meat, or other animal products, and maintaining acceptable levels of milk components. A healthy rumen environment reduces the risk and incidence of metabolic disorders that can occur with improper feeding management or abrupt feed intake changes, such as acidosis.

Current cattle industry practices incorporate the genetic selection of certain characteristics such as feed efficiency or maximized milk production. Genetic selection can result in animals that gain weight efficiently and/or produce large volumes of high quality milk. Genetically selected cattle are fed energy dense rations to maximize milk production. Energy dense rations can inhibit fiber fermentation and increase the difficulty of maintaining an optimal rumen environment. The feeding of high concentrate diets for cattle has increased awareness of problems associated with ruminal acidosis and the implications it has on feed consumption, feed efficiency, production of milk, meat, or other animal products, and animal health.

Current practices also include self-feeding for certain animals, which may include finishing beef and finishing dairy beef cattle. Self-fed nutrition management programs provide all or part of an animal's complete diet in feeder equipment that allows free access for the animal to this diet at all times of the day over the entire feeding period. Self-feeding often occurs in dry lot environments, but can occur in other environments as well. Typically, self-fed programs are designed for feeding beef and dairy cattle to the appropriate weight and fatness level whereby the resulting meat satisfies the standards of the meat industry. Self-fed programs can also be provided at any time during the growing and development periods for animals to achieve their desired weight and maturity. Because animals lack the nutritional wisdom to regulate their feed consumption to optimize health, growth, and beneficial rumen microbial growth, acute and subacute digestive disorders can result in these self-fed environments.

A substantial drop in ruminal pH can damage the rumen wall, decrease blood pH, cause dehydration, laminitis, or erratic feed consumption patterns, and severely depress feed intake and feed efficiency. During acidosis and the recovery period, females may also experience decreased milk production. Maintaining a healthy rumen pH and preventing disorders such as acidosis can therefore improve feed intake, feed efficiency, productive milk output, milk quality indices, and feedlot animal performance, including meat production.

One aim of the present invention is to provide a feed supplement which provides improved availability of at least one nutrient or an improvement in the energy gained from a feed material.

### BRIEF SUMMARY OF THE INVENTION

It is the object of the present invention to provide compositions and methods for improving livestock health and performance. In one aspect, the compositions and methods produce improvements in feed intake, feed efficiency, productive milk output, milk quality indices, and/or meat production.

The novel compositions of the present invention are intended to be added to a conventional livestock feed, although they can be given alone. The novel compositions are formulated substantially according to any of Tables 1-4. The supplement can be added to the feed is in the form of a powder, liquid, or solid. The supplement may also be provided as, or integrated into, a feed block. The compositions may be otherwise administered orally. The oral administration of enhanced feed in the form of a liquid or suspension also presents numerous additional benefits to the bovine producers. For example, benefits are seen as a result of increased ease of application and use, decreased cost and improved convenience of administration.

In another aspect, the compositions of the present invention can be used for improving the ruminal health of a ruminant animal. Improvements in ruminal health through use of the compositions can include, for example, increased nutrient availability and absorption by the animal, increased fermentation, and increased rumen microbial growth.

There are also numerous additional benefits to the quality of the meat derived from cattle provided with the compositions of the present invention. These benefits are derived in addition to the significant benefits seen in increasing the health and performance of the livestock.

### DETAILED DESCRIPTION OF THE INVENTION

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, processes and examples described in the description of the invention are illustrative only and not intended to be limiting to the scope of the invention in any manner. Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims.

The present invention relates to improved health and performance of livestock through provision of compositions, including compositions provided as a food supplement or additive. Applicant provides a special enrichment diet for beef cattle providing numerous benefits, including, for example, improved muscle mass and tone, fertility, hair growth, eye health, feed intake, feed efficiency, productive milk output, milk quality indices, meat production, and/or reduced morbidity and mortality.

In a preferred embodiment of the methods of the present invention for feeding livestock, the health and performance of livestock can be enhanced by feeding the composition in an amount of, for example, at least 1 oz. per head, per day to cattle less than 800 lbs, and 2 oz. per head, per day to cattle weighing more than 800 lbs. The composition may be provided for a limited period of time, or may be provided over the lifetime of the animal.

The composition may include sodium bentonite, sodium sulfate, dried Aspergillus oryzae fermentation product, ferrous sulfate, calcium carbonate, manganese proteinate, manganese sulfate, molasses products, vegetable oil, lecithin, high fructose corn syrup, polysorbate 60, glycerin, sulfuric acid, xanthan gum, natural and artificial flavor, zinc proteinate, selenium yeast, zinc sulfate, yeast culture, vitamin A supplement, vitamin D-3 supplement, vitamin E supplement, choline chloride, niacin, calcium pantothenate, riboflavin, vitamin B-12 supplement, thiamine hydrochloride, copper sulfate, copper proteinate, Lactobacillus acidophilus, Lactobacillus brevis, Lactobacillus plantarum, Enterococcus faecium, and Pediococcus acidlacticii. A further preferred feeding range consists of feeding beef cattle throughout its life with the special enriched diet. The dietary composition can be further enriched with or provided in addition to organic trace minerals and vitamins.

It is understood that the compositions, i.e. dietary supplements and additives, can be provided alone or mixed into feed. For example, the compositions may be mixed into daily ration at a rate of 1 oz. per head per day to cattle less than 800 lb. Feed 2 oz per head per day to cattle weighing more than 800 lb. The dietary supplements or additives according to the present invention can be formulated substantially according to any of Tables 1-4, below.

TABLE 1
Exemplary ingredient solution.

| Ingredient | Amount (lbs.) |
|---|---|
| Sodium Bentonite | 820.5 |
| Sodium sulfate | 550 |
| Limestone | 237.8 |
| Diamond V XP Yeast | 125 |
| Whey permeate | 50 |
| cs Iron (trouwe) | 45 |
| Corn oil | 33 |
| Zinc Proteinate (trouwe) | 33 |
| Manganous oxide | 33 |
| SAMI 5X VITES (Vitamin mix - A, D, and E) | 27 |
| Selenium | 20.2 |
| Ascorbic acid | 20 |
| Min Rich C&S Rev | 20 |
| Vitamin E (227000 IU/lb) | 2.5 |
| E.D.D.I. (Iodine) | 1 |
| BioPlus2B | 1 |
| Flavor | 1 |
| Choline (60% dry) | 0.5 |
| Vitamin B12 (4540 mg) | 0.5 |
| Amaferm | 3 |
| Total | 2024 |

TABLE 2
Exemplary nutrient solution.

| calcium | 5.81% | |
|---|---|---|
| Phosphorus | .659% | |
| Phos; avail | .657% | |
| Salt | .018% | |
| Sodium | .774% | |
| Potassium | .188% | |
| Sulfur | .098% | |
| Magnesium | .045% | |
| Chloride | .005% | |
| manganese | 10379 | ppm |
| Zinc | 2286.23 | ppm |
| Iron | 4918.51 | ppm |
| Copper | 99.91 | ppm |
| Selenium | 2.008 | ppm |
| Vitamin A | 40.205 | KIU/lb. |
| Vitamin A; stb. Eq | 40.020 | KIU/lb |
| Vitamin E | 414.526 | IU/lb |
| Menadione | 53.360 | mg/lb |

TABLE 3

| | |
|---|---|
| Sodium Bentonite | 40.54% |
| Sodium Sulfate | 27.17% |
| Dried Aspergillus oryzae Fermentation | 7.05% |
| Product | |
| Ferrous Sulfate | 6.65% |
| Calcium Carbonate | 5.62% |
| Manganese Proteinate | 2.80% |
| Manganese Sulfate | 1.96% |
| Molasses/Vegtable Oil Blend | 1.63% |
| Zinc Proteinate | 1.63% |
| Selenium Yeast | 1.35% |
| Zinc Sulfate | 1.04% |
| Yeast Culture | .99% |
| RAF Beef Blue (B-Vitamins) | .67% |
| Copper Sulfate | .54% |
| Copper Proteinate | .21% |
| Vitamin E 50% | .09% |
| Life 10-G Microbial | .05% |

TABLE 4
Exemplary formulation - vitamin, mineral for beef cattle fed in confinement for
slaughter. For improved feed efficiency.

| | | | | | | |
|---|---|---|---|---|---|---|
| Calcium (Ca) | (Min) | 2.0% | Vitamin A | (Min) | 396,000 | IU/lb |
| Calcium (Ca) | (Max) | 2.5% | Vitamin D-3 | (Min) | 41,000 | IU/lb |
| Sodium (Na) | (Min) | 3.6% | Vitamin E | (Min) | 400 | IU/lb |
| Sodium (Na) | (Max) | 4.0% | Vitamin B-12 | (Min) | 17.2 | mcg/lb |
| Copper (Cu) | (Min) | 1,590 ppm | Riboflavin | (Min) | 6.0 | mg/lb |
| Iron (Fe) | (Min) | 19,150 ppm | d-Pantothenic Acid | (Min) | 10.0 | mg/lb |
| Manganese | (Min) | 9,970 ppm | Thiamine | (Min) | 1 | mg/lb |
| (Mn) | | | | | | |
| Selenium (Se) | (Min) | 26.8 ppm | Niacin | (Min) | 60.0 | mg/lb |
| Zinc (Zn) | (Min) | 5,780 ppm | Choline | (Min) | 240.0 | mg/lb |

The results of feeding the special enhanced diet of the present invention to beef cattle illustrate that cattle exhibiting stress or inability to thrive (i.e., foundering calves) exhibit substantial improvements in health and performance following administration of dietary supplements according to the present invention. The improvements include increases in muscle tissue, hair growth, fertility, feed intake, feed efficiency, productive milk output, milk quality indices, and/or meat production. There is evidence suggesting the dietary supplements can be used both to improve the performance and health of livestock not exhibiting any disease or defect, and to treat livestock exhibiting defects and/or disease symptoms, including, for example, poor growth or failure to thrive.

Further, the formulations according to the present invention can also be used for livestock other than cattle. However, it should be understood that the formulae may require modification for administration for certain livestock. For example, removal of copper is required before administration of sheep.

The invention has been described with reference to various specific and preferred embodiments and techniques. However, it should be understood that many variations and modifications may be made while remaining within the spirit and scope of the invention. In particular, it is to be understood that the present invention contemplates variations in the organic components of the special enriched diet, process parameters for making the special enriched diet, including temperature, time, pH, salts, manufacturing techniques, drying or preparation techniques, and selenoproteins of interest.

All publications in this specification are indicative of the level of ordinary skill in the art to which this invention pertains. All publications are herein incorporated by reference in its entirety to the same extent as if each individual publication was specifically and individually indicated by reference.

What is claimed is:

1. A dietary supplement according substantially to the formulation of any of Tables 1-4.

2. The dietary supplement according to claim 1, wherein said supplement is in the form of a powder, liquid, or solid.

3. The dietary supplement according to claim 1, wherein said supplement is provided as, or integrated into, a feed block.

4. A method of improving livestock health and performance comprising providing to said livestock a dietary supplement substantially according to the formulation of any of Tables 1-4.

5. The method of claim 4 wherein said improvement comprises one or more of fertility, hair growth, eye health, feed intake, feed efficiency, productive milk output, milk quality indices, and/or meat production.

6. The method according to claim 4 wherein said supplement is in the form of a powder, liquid, or solid.

7. The method according to claim 4 wherein said supplement is provided as, or integrated into, a feed block.

8. The method according to claim 4 wherein said livestock are cattle.

9. The method according to claim 4 wherein said providing comprises at least 1 oz. per head, per day to cattle less than 800 lbs, and 2 oz. per head, per day to cattle weighing more than 800 lbs.

10. A feed composition for livestock comprising an additive or supplement substantially according to the formulations of any of Tables 1-4.

11. The composition according to claim 10, wherein said additive or supplement is in the form of a powder, liquid, or solid.

12. The composition according to claim 10 wherein said additive or supplement is provided as, or integrated into, a feed block.

13. A livestock animal with improved health and performance, produced by a method of providing said animal with a composition substantially according to the formulations of any of Tables 1-4.

14. The livestock animal according to claim 13 wherein said animal is bovine.

15. The livestock animal according to claim 13 wherein said composition is in the form of a powder, liquid, or solid.

16. The livestock animal according to claim 13 wherein said composition is provided as, or integrated into, a feed block.

17. The livestock animal according to claim 13 wherein said providing comprises at least 1 oz. per head, per day to cattle less than 800 lbs, and 2 oz. per head, per day to cattle weighing more than 800 lbs.

18. A method of improving ruminal health in a ruminant animal comprising providing said animal with a composition substantially according to the formulations of any of Tables 1-4.

19. The method according to claim 18 wherein said composition is provided as, or integrated into, a feed block.

20. The method according to claim 18 wherein said providing comprises at least 1 oz. per head, per day to cattle less than 800 lbs, and 2 oz. per head, per day to cattle weighing more than 800 lbs.
